(19) United States
(12) Patent Application Publication
(10) Pub. No.: US 2004/0131670 A1
(43) Pub. Date: Jul. 8, 2004

(54) Pellicle-resistant gelatin capsule

(76) Inventor: Ping Gao, Portage, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/633,102

(22) Filed: Jul. 31, 2003

Related U.S. Application Data
(63) Continuation-in-part of application No. 10/119,129, filed on Apr. 9, 2002.
(60) Provisional application No. 60/284,381, filed on Apr. 17, 2001.
(60) Provisional application No. 60/326,952, filed on Oct. 4, 2001.
(60) Provisional application No. 60/399,862, filed on Jul. 31, 2002.
(60) Provisional application No. 60/399,776, filed on Jul. 31, 2002.
(60) Provisional application No. 60/399,863, filed on Jul. 31, 2002.
(60) Provisional application No. 60/399,808, filed on Jul. 31, 2002.

(51) Int. Cl. A61K 9/64

(52) U.S. Cl. 424/456

(57) ABSTRACT

The present invention relates to compositions suitable for use in preparing gelatin capsules, to gelatin capsules exhibiting reduced cross-linking, and to methods of preparing such gelatin capsules.

This application is a continuation-in-part of U.S. application Ser. No. 10/119,129 filed on 09 Apr. 2002, which claims priority of U.S. provisional application Serial No. 60/284,381 filed on 17 Apr. 2001 and U.S. provisional application Serial No. 60/326,952 filed on 04 Oct. 2001. This application also claims priority of U.S. provisional application Serial No. 60/399,862 filed on 31 Jul. 2002, U.S. provisional application Serial No. 60/399,776 filed on 31 Jul. 2002, U.S. provisional application Serial No. 60/399,863 filed on 31 Jul. 2002, and U.S. provisional application Serial No. 60/399,808 filed on 31 Jul. 2002.

### FIELD OF THE INVENTION

The present invention relates to gelatin capsules which exhibit reduced gelatin cross-linking. Such capsules are useful in, inter alia, the pharmaceutical, nutraceutical, and food industries.

### BACKGROUND OF THE INVENTION

Gelatin, a mixture of water-soluble proteins derived from collagen by hydrolysis, is widely used in the pharmaceutical and food industries, among others. One major application of gelatin is in preparation of both hard and soft gelatin capsules. Such capsules are desirable for, inter alia, their versatility (they may contain drug formulations in solid, semi-solid, or liquid form) and for their rapid dissolution characteristics. Unfortunately, drug dosage forms containing gelatin in an outer layer (e.g. liquid or powder filled into a gelatin capsule) are known to exhibit a drop in dissolution rate over time. This drop in dissolution rate can lead to undesirable and unacceptable alterations in in vitro dissolution profile and in bioavailability, particularly for drugs of low water solubility or drugs whose absorption is dissolution-rate limited. Such changes in dissolution profile are thought to result from cross-linking of gelatin occurring in capsule shells.

Singh et al., Alteration in Dissolution Characteristics of Gelatin-Containing Formulations, Pharmaceutical Technology, April 2002, hereby incorporated by reference herein but not admitted to be prior art, describes reports suggesting that several agents including glycerine, glycine, and hydroxylamine hydrochloride, when incorporated into fill contents of gelatin capsules, can limit gelatin cross-linking.

Unfortunately, existing methods directed at the problem of gelatin cross-linking in capsule shells are less than satisfactory, particularly in situations where longer shelf life and stability through real life storage, shipping and handling conditions are desired; pursuit of adequate solutions to the problem of gelatin capsule cross-linking is therefore desirable.

If gelatin capsules could be prepared which are capable of providing a predictable and stable dissolution rate of a drug contained therein, even after storage of such capsules under stressed conditions, a significant advance in the oral delivery of drugs, particularly drugs of low water solubility or drugs whose absorption is dissolution-rate limited, would result.

### SUMMARY OF THE INVENTION

There is now provided a composition suitable for preparing a pharmaceutical capsule shell, the composition comprising gelatin and an amine agent that comprises at least one pharmaceutically acceptable primary or secondary amine. Desirably, the amine agent is present in an amount effective to inhibit cross-linking of the gelatin and/or pellicle formation in a capsule shell prepared from the composition.

There is further provided a capsule shell of the instant composition.

There is still further provided a pharmaceutical dosage form comprising capsule shells of the instant composition, wherein the shells define a fill volume that is at least partially occupied by a fill material. The fill material preferably contains a drug, more preferably a drug of low water solubility.

In one embodiment, the drug is a selective cyclooxygenase-2 inhibitory drug.

The composition and dosage form of the present invention are especially useful for liquid fill materials and for soft gelatin capsules.

The term “pellicle” herein refers to a relatively water-insoluble membrane formed in a gelatin capsule shell. Such a membrane tends to be thin, tough, and rubbery. It is now understood that one mechanism underlying pellicle formation is gelatin cross-linking. Gelatin cross-linking and pellicle formation result in reduced dissolution rates. Accordingly, quantification of dissolution rate of a first capsule within a reasonably short time after capsule preparation and of a second capsule after storage under stressed conditions (e.g., four weeks at 40° C. and 85% relative humidity in a closed container) as described herein provides one means of assessing pellicle formation and/or gelatin cross-linking. The term “within a reasonably short time after capsule formation” means within a period such that substantial cross-linking and/or pellicle formation is unlikely to have yet occurred, for example within one week, dependent upon storage conditions during that period.

The term “pellicle-resistant” herein means that a gelatin capsule so described has a reduced tendency to form, or exhibits slowed, delayed or reduced formation of a pellicle upon storage under stressed conditions. Similarly, “inhibition of cross-linking” (or “inhibition of pellicle formation”) herein means a slowed, delayed or reduced formation of gelatin cross-links (or pellicle formation) in a capsule by comparison with an otherwise similar capsule lacking only the amine agent as provided herein.

Pharmaceutical dosage forms according to the present invention have been found to exhibit an unexpected and surprisingly substantial reduction in cross-linking of gelatin in the capsule shell and in pellicle formation. As a result, such dosage forms are capable of consistently meeting desired in vitro dissolution criteria, even after storage under stressed conditions. This invention represents a significant improvement over conventional dosage forms and conventional gelatin capsule shells.

### BRIEF DESCRIPTION OF THE DRAWINGS

FIG. 1 is a graph showing Tier I dissolution rate of Formulation 30 following storage at 25° C. as described in Example 6.

FIG. 2 is a graph showing Tier I dissolution rate of Formulation 30 following storage at 40° C. as described in Example 6.

FIG. 3 is a graph showing Tier II dissolution rate of Formulation 30 following storage at 25° C. as described in Example 6.

FIG. 4 is a graph showing Tier II dissolution rate of Formulation 30 following storage at 40° C. as described in Example 6.

FIG. 5 is a graph showing Tier I dissolution rate of Formulation 19 following storage at 25° C. as described in Example 6.

FIG. 6 is a graph showing Tier I dissolution rate of Formulation 19 following storage at 40° C. as described in Example 6.

FIG. 7 is a graph showing Tier II dissolution rate of Formulation 19 following storage at 40° C. as described in Example 6.

### DETAILED DESCRIPTION OF THE INVENTION

Composition Suitable For Preparation of a Capsule Shell

In one embodiment, the present invention provides a composition suitable for preparation of a capsule shell. Such a composition, according to the present invention, comprises gelatin and an amine agent comprising at least one pharmaceutically acceptable primary or secondary amine present in an amount effective to inhibit cross-linking of the gelatin shell and/or pellicle formation upon storage.

The term primary or secondary amine compound herein includes those primary and secondary amine compounds which are pharmaceutically acceptable excipients. Preferably, primary and secondary amine compounds of the instant invention are not therapeutically or nutritionally active compounds.

Prior to using such a composition in preparation of gelatin capsules shells, a liquid, for example water, is typically added to the composition to form an aqueous mixture. In one embodiment, such a composition suitable for preparation of a capsule shell comprises gelatin, at least one primary or secondary amine compound, and water. Preferably, water will be present in an amount such that the weight ratio of water to gelatin is about 0.8 to about 1.6, and preferably about 1 to about 1.3.

Preferably, gelatin is present in a composition of the invention in an amount of about 1% to about 99%, more preferably about 10% to about 80%, and still more preferably about 15% to about 90% of the composition on a dry weight basis. It should be understood that “on a dry weight basis” means total weight excepting water weight.

Amines of the Composition.

Any pharmaceutically acceptable primary or secondary amine can be used in a composition of this embodiment. Non-limiting examples of suitable primary amines include tris(hydroxymethyl)aminomethane (also known and/or referred to herein as tromethamine or Tris), ethanolamine, ethylenediamine, diethylamine, ethylene N-methyl-D-glucamine, and amino acids such as L-arginine, L-lysine, and guanidine. Non-limiting examples of suitable secondary amines include diethanolamine, benethamine (i.e., N-phenymethyl)benezeneethanamine), benzathine (i.e., N,N-dibenzylethylenediamine), piperazine, hydrabamine (i.e., N,N-bis(dehydroabietyl)ethylenediamine), and imidazole. The amine compound is preferably present in a composition of the invention in a total amount of not more than about 10%, for example about 0.01% to about 10%, preferably about 0.1% to about 5%, and more preferably about 0.1% to about 2% of the composition on a dry weight basis.

Optional Excipients of the Composition.

A composition of the invention, in addition to gelatin and a primary or secondary amine, preferably further comprises one or more pharmaceutically acceptable excipients. For example, where a composition of the invention is to be used to prepare soft gelatin capsules, the composition preferably comprises at least one plasticizer in a total amount of about 2% to about 60%, preferably about 5% to about 45%, and more preferably about 10% to about 40% of the composition on a dry weight basis. Where a plasticizer is present, the weight ratio of plasticizer (dry weight) to gelatin is about 0.3 to about 1.8 and preferably about 0.4 to about 0.75. Non-limiting examples of suitable plasticizers include poly-hydroxy-alcohols such as sorbitol, glycerol, and mannitol; dialkylphthalates; lower alkyl citrates wherein the lower alkyl has 1-6 carbon atoms; glycols and polyglycols including polyethylene glycols with a molecular weight range of about 200 to about 40,000, methoxyl-propylene-glycol, and 1,2-propylene glycol; esters of polyhydroxy-alcohols such as mono-, di-, and tri-acetate of glycerol; ricinoleic acid and esters thereof; and mixtures of the above.

A composition of the invention can further comprise one or more preservatives, opacifying agents (e.g. titanium dioxide), decomposition inhibitors (e.g. sulfur dioxide) color, flavor, etc. Non-limiting illustrative examples of suitable preservatives include methylparaben, propylparaben, butylparaben, sorbic acid, benzoic acid, editic acid, phenolic acids, potassium sorbate, and sodium propionate.

Optionally, a composition of the invention further comprises at least one pharmaceutically acceptable sulfite compound. Illustrative pharmaceutically acceptable sulfite compounds include sodium metabisulfite, sodium bisulfite, and sodium thiosulfate (sodium hyposulfite). If present, one or more sulfite compounds are preferably present in a composition of the invention in an amount of not more than about 10%, for example about 0.01% to about 5%, and preferably about 0.1% to about 2% of the composition on a dry weight basis.

A composition suitable for preparation of a capsule wall can be in the form of a solid, a dry powder, a semi-solid, a liquid solution, or a liquid suspension. Importantly, a suitable physical form (e.g. powder, liquid mixture, etc.) of a composition of the invention will, at least in part, be determined by the particular process, if any, which will be used to make capsules, and by the particular type of capsule being prepared (hard or soft). One of ordinary skill in the art will readily select a suitable physical form for a composition of the invention taking these and other factors into consideration.

Process For Preparing Capsules of the Invention

i. Hard Capsules

A composition of the invention can be used to prepare hard gelatin capsules according to any suitable process including but not limited to those processes described in the following patents and/or publications, each of which is hereby incorporated by reference.

U.S. Pat. No. 3,656,997 to Cordes.

U.S. Pat. No. 4,231,211 to Strampfer et al.

U.S. Pat. No. 4,263,251 to Voegle.

U.S. Pat. No. 4,403,461 to Goutard et al.

U.S. Pat. No. 4,705,658 to Lukas.

U.S. Pat. No. 4,720,924 to Hradecky et al.

U.S. Pat. No. 4,756,902 to Harvey et al.

U.S. Pat. No. 4,884,602 to Yamamoto et al.

U.S. Pat. No. 4,892,766 to Jones.

U.S. Pat. No. 6,350,468 to Sanso.

International Patent Publication No. WO 84/00919 to Mackie.

International Patent Publication No. WO 85/04100 to Kalidindi.

One of ordinary skill in the art will readily adapt the processes described in the above documents in view of the present disclosure in order to prepare capsules comprising an amine compound according to the present invention.

One preferred method for preparing hard gelatin capsules of the invention comprises the steps of (a) providing a composition suitable for preparation of a capsule shell (comprising gelatin and a primary or secondary amine compound) in dry powder form, (b) preparing a liquid solution or solution/suspension comprising water and the composition, (c) heating the liquid, (d) dipping stainless steel capsule-making pins in the heated liquid, (e) removing the dipped pins from the liquid to form coated pins, and (f) subjecting the coated pins to a drying process to produce a dry capsule half-shell. After drying, capsule half-shells are removed from the pins and trimmed to desired length. The capsule half-shells can next be filled with any desired fill material, brought together in a cooperative manner to form a capsule shell, and then capped. Optionally, the cap can be spot welded, fused or banded with molten gelatin to provide a tamper-resistant product. According to this process, the primary or secondary amine compound is preferably present in the composition suitable for making a capsule shell, and/or if desired, can also be added during steps (b), (c) and/or (d).

ii. Soft Capsules

Soft gelatin capsules of the invention can be prepared according to any suitable process including but not limited to the plate process, vacuum process, or the rotary die process. See, for example, (1) Ansel et al. (1995) in Pharmaceutical Dosage Forms and Drug Delivery Systems, 6th ed., Williams & Wilkins, Baltimore, Md., pp. 176-182; and (2) Remington: The Science and Practice of Pharmacy, 19th Ed., Mack Publishing Co. Easton. Pa., pp. 1646-1647, the above-recited pages of which are hereby incorporated by reference herein.

The rotary die process is a presently preferred process by which to make soft gelatin capsules according to the present invention. According to the rotary die process, a composition of the invention comprising gelatin and a primary or secondary amine compound is dissolved or suspended in water to form a flowable material and is then placed in an overhead tank. The flowable material from the overhead tank is formed into two continuous ribbons by a rotary die machine and the ribbons are then brought together by twin rotating dies. Simultaneously, metered fill material is injected between ribbons at approximately the same moment that the dies form pockets of the ribbons. These pockets of fill-containing encapsulation material are then sealed by pressure and heat.

Soft gelatin capsules can be manufactured in different shapes including round, oval, oblong, and tube-shape, among others. Additionally, by using two different ribbon colors, two-tone capsules can be produced.

Non-limiting examples of suitable methods for preparing soft gelatin capsules are described in the following patents and publications, each of which is hereby incorporated by reference.

U.S. Pat. No. 3,592,945 to Pesch.

U.S. Pat. No. 4,609,403 to Wittwer et al.

U.S. Pat. No. 4,744,988 to Brox.

U.S. Pat. No. 4,804,542 to Fischer et al.

U.S. Pat. No. 5,146,758 to Herman.

U.S. Pat. No. 5,254,294 to Wunderlich et al.

U.S. Pat. No. 6,260,332 to Takayanagi.

U.S. Pat. No. 6,238,616 to Ishikawa et al. and

International Patent Publication No. WO 92/15828 to Herman.

As used herein, unless specific context instructs otherwise, the term “capsule shell” (and “gelatin capsule shell”) embraces capsule half-shells (that can cooperate to form a whole capsule shell) and whole capsule shells (that define a fill volume). Such term also embraces soft gelatin capsule shells and hard gelatin capsules, irrespective of the process by which such shells are made.

The terms “sealed capsule shell”, “sealed in a capsule shell”, “sealing in the capsule shell” and the like are meant to denote a whole capsule shell that defines a fill volume, that such fill volume can contain a fill material, that such fill material is enclosed in the whole capsule shell, and that such enclosure affords the fill material more than a de minimus amount of protection from the atmosphere outside of the whole capsule shell.

Drug of Low Water Solubility.

Capsule shells according to the present invention define a fill volume and such fill volume can be occupied at least partially by any fill material. The fill material can comprise any active drug. Preferably, the active drug is a drug of low water solubility, also referred to herein as a poorly water soluble drug. A “drug of low water solubility” or “poorly water solubility drug” herein refers to any drug or compound having a solubility in water, measured at 37° C., not greater than about 10 mg/ml, and preferably not greater than about 1 mg/ml. Particularly preferred drugs having a solubility in water, measured at 37° C., not greater than about 0.1 mg/ml.

Solubility in water for many drugs can be readily determined from standard pharmaceutical reference books, for example The Merck Index, 11th ed., 1989 (published by Merck & Co., Inc., Rahway, N.J.); the United States Pharmacopoeia, 24th ed. (USP 24), 2000; The Extra Pharmacopoeia, 29th ed., 1989 (published by Pharmaceutical Press, London); and the Physicians Desk Reference (PDR), 2001 ed. (published by Medical Economics Co., Montvale, N.J.).

For example, individual drugs of low solubility as defined herein include those drugs categorized as “slightly soluble”, “very slightly soluble”, “practically insoluble” and “insoluble” in USP 24, pp. 2254-2298; and those drugs categorized as requiring 100 ml or more of water to dissolve 1 g of the drug, as listed in USP 24, pp. 2299-2304.

Illustratively, suitable drugs of low water solubility include, without limitation, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelminthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

Non-limiting illustrative examples of suitable drugs of low water solubility include acetohexamide, acetylsalicylic acid, alclofenac, allopurinol, atropine, benzthiazide, carprofen, celecoxib, chlordiazepoxide, chlorpromazine, clonidine, codeine, codeine phosphate, codeine sulfate, deracoxib, diacerein, diclofenac, diltiazem, estradiol, etodolac, etoposide, etoricoxib, fenbufen, fenclofenac, fenprofen, fentiazac, flurbiprofen, griseofulvin, haloperidol, ibuprofen, indomethacin, indoprofen, ketoprofen, lorazepam, medroxyprogesterone acetate, megestrol, methoxsalen, methylprednisone, morphine, morphine sulfate, naproxen, nicergoline, nifedipine, niflumic, oxaprozin, oxazepam, oxyphenbutazone, paclitaxel, phenindione, phenobarbital, piroxicam, pirprofen, prednisolone, prednisone, procaine, progesterone, pyrimethamine, rofecoxib, sulfadiazine, sulfamerazine, sulfisoxazole, sulindac, suprofen, temazepam, tiaprofenic acid, tilomisole, tolmetic, valdecoxib, etc.

The amount of drug incorporated into fill material to be filled into a capsule of the invention can be selected according to known principles of pharmacy. A therapeutically effective amount of drug is specifically contemplated. The term “therapeutically and/or prophylactically effective amount” as used herein refers to an amount of drug that is sufficient to elicit the required or desired therapeutic and/or prophylactic response. Preferably, the therapeutic agent is present in an amount of at least about 0.01%, preferably at least about 0.1%, more preferably at least about 1%, and still more preferably at least about 5%, by weight of the composition on a dry weight basis.

Selective COX-2 Inhibitory Drugs.

In a preferred embodiment, the drug is a selective cyclooxygenase-2 inhibitory drug. A preferred selective cyclooxygenase-2 inhibitory drug useful herein, or to which a salt or prodrug useful herein is converted in vivo, is a compound of formula (I)

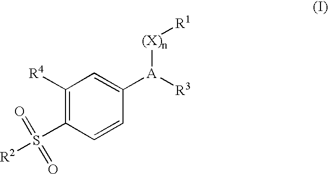

wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is 0 or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

Compositions of the invention are especially useful for selective cyclooxygenase-2 inhibitory drugs having the formula (II):

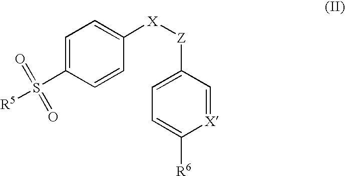

where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Illustratively, capsules of the invention are suitable for delivering celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, pharmaceutically acceptable salts and prodrugs thereof.

Capsules of the invention are also useful for compounds having the formula (III):

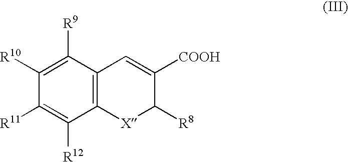

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy, or aryl; and for pharmnaceutically acceptable salts thereof.

A particularly useful compound of formula (III) is (S)-6,8-dichloro-2-(trifluoromethyl)-2H-1-benzopyran-3-carboxylic acid, particularly in the form of a water-soluble salt thereof, for example the sodium salt.

The invention is illustrated herein with particular reference to celecoxib, and it will be understood that any other selective COX-2 inhibitory drug can, if desired, be substituted in whole or in part for celecoxib in dosage forms herein described. For example, dosage forms of the invention are suitable for formulation of valdecoxib, alone or in combination with celecoxib.

Where the drug is celecoxib, the dosage form typically comprises celecoxib in a therapeutically and/or prophylactically effective total amount of about 10 mg to about 1000 mg per dose unit. Where the drug is a selective COX-2 inhibitory drug other than celecoxib, the amount of the drug per dose unit is therapeutically equivalent to about 10 mg to about 1000 mg of celecoxib.

It will be understood that a therapeutically and/or prophylactically effective amount of a drug for a subject is dependent inter alia on the body weight of the subject. A “subject” herein to which a therapeutic agent or composition thereof can be administered includes a human patient of either sex and of any age, and also includes any nonhuman animal, particularly a domestic or companion animal, illustratively a cat, dog or horse.

Where the subject is a child or a small animal (e.g., a dog), for example, an amount of celecoxib relatively low in the preferred range of about 10 mg to about 1000 mg is likely to be consistent with therapeutic effectiveness. Where the subject is an adult human or a large animal (e.g., a horse), therapeutic effectiveness is likely to require dose units containing a relatively greater amount of celecoxib. For an adult human, a therapeutically effective amount of celecoxib per dose unit in a dosage form of the present invention is typically about 10 mg to about 400 mg. Especially preferred amounts of celecoxib per dose unit are about 100 mg to about 200 mg, for example about 100 mg or about 200 mg.

For other selective COX-2 inhibitory drugs, an amount of the drug per dose unit can be in a range known to be therapeutically effective for such drugs. Preferably, the amount per dose unit is in a range providing therapeutic equivalence to celecoxib in the dose ranges indicated immediately above.

Liquid Filled Capsule

In a preferred embodiment, a capsule of the invention is filled with a liquid fill material. More preferably, the fill material is self-emulsifying upon contact with simulated gastric fluid. Fill material according to this embodiment comprises at least one solvent which is preferably suitable for dissolving the drug and/or any additional ingredients or excipients present therein.

i. Glycol Solvents

A preferred liquid solvent is a glycol or glycol ether. Suitable glycol ethers include those conforming to formula (X):

R¹—O—((CH₂)ₘO)ₙ—R² (X)

wherein R¹ and R² are independently hydrogen or C₁₋₆ alkyl, C₁₋₆ alkenyl, phenyl or benzyl groups, but no more than one of R¹ and R² is hydrogen; m is an integer of 2 to about 5; and n is an integer of 1 to about 20. It is preferred that one of R¹ and R² is a C₁₋₄ alkyl group and the other is hydrogen or a C₁₋₄ alkyl group; more preferably at least one of R¹ and R² is a methyl or ethyl group. It is preferred that m is 2. It is preferred that n is an integer of 1 to about 4, more preferably 2.

Glycol ethers used as solvents in fill material typically have a molecular weight of about 75 to about 1000, preferably about 75 to about 500, and more preferably about 100 to about 300. Importantly, the glycol ethers used in fill material of this embodiment must be pharmaceutically acceptable and must meet all other conditions prescribed herein.

Non-limiting examples of glycol ethers that may be used in fill material of this embodiment include ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof. See for example Flick (1998): Industrial Solvents Handbook, 5th ed., Noyes Data Corporation, Westwood, N.J. A particularly suitable glycol ether solvent is diethylene glycol monoethyl ether, sometimes referred to in the art as DGME or ethoxydiglycol. It is available for example under the trademark Transcutol™ of Gattefosse Corporation.

Glycols suitable as solvents in fill material include propylene glycol, 1,3-butanediol and polyethylene glycols. A presently preferred solvent is polyethylene glycol (PEG).

Any pharmaceutically acceptable PEG can be used. Preferably, the PEG has an average molecular weight of about 100 to about 10,000, and more preferably about 100 to about 1,000. Still more preferably, the PEG is of liquid grade. Non-limiting examples of PEGs that can be used in solvent liquids of this invention include PEG-200, PEG-350, PEG-400, PEG-540 and PEG-600. See for example Flick (1998), op. cit., p. 392. A presently preferred PEG has an average molecular weight of about 375 to about 450, as exemplified by PEG-400.

Polyethylene glycols such as PEG-400 have many desirable properties as solvents for poorly water-soluble drugs. In the case of celecoxib, for example, the drug can be dissolved or solubilized at a very high concentration in PEG-400, enabling formulation of a therapeutically effective dose in a very small volume of solvent liquid. This is especially important where the resulting solution is to be encapsulated, as capsules of a size convenient for swallowing can be prepared containing a therapeutically effective dose even of a drug such as celecoxib having a relatively high dose requirement for efficacy. Importantly, ethanol, water, and other excipients identified as co-solvents hereinbelow or elsewhere can, if desired, be used as solvents in a fill material of the invention. Typically, one or more solvents will be present in a fill material in a total amount of about 5% to about 95%, preferably about 10% to about 90% and more preferably about 15% to about 85%, by weight of the fill material.

ii. Co-Solvents

A fill material of this embodiment optionally comprises one or more pharmaceutically acceptable co-solvents. Non-limiting examples of suitable co-solvents include additional glycols, alcohols, for example ethanol and n-butanol; oleic and linoleic acid triglycerides, for example soybean oil; caprylic/capric triglycerides, for example Miglyol™ 812 of Huls; caprylic/capric mono- and diglycerides, for example Capmul™ MCM of Abitec; polyoxyethylene caprylic/capric glycerides such as polyoxyethylene (8) caprylic/capric mono- and diglycerides, for example Labrasol™ of Gattefosse; propylene glycol fatty acid esters, for example propylene glycol laurate; polyoxyethylene (35) castor oil, for example Cremophor™ EL of BASF; polyoxyethylene glyceryl trioleate, for example Tagat™ TO of Goldschmidt; lower alkyl esters of fatty acids, for example ethyl butyrate, ethyl caprylate and ethyl oleate; and water.

Excipient Fill Material.

Sulfite Compound.

Where a pharmaceutical dosage form is to be provided, fill material placed into a capsule of the present invention can further comprise a sulfite compound. In a preferred embodiment, at least about 40%, preferably at least about 50%, still more preferably at least about 55%, even more preferably at least about 60%, and yet more preferably at least about 70% of all sulfite compound present in a dosage unit of the invention is present in the fill material.

Amine Agent.

Where a pharmaceutical dosage form is to be provided, fill material (which is placed into a capsule according to the present invention) can further comprise a primary or secondary amine compound. However, in a particularly preferred embodiment, at least about 40%, preferably at least about 50%, still more preferably at least about 55%, even more preferably at least about 60%, and yet more preferably at least about 70% of all primary or secondary amine compound present in a dosage unit of the invention is present in the capsule shell.

Sulfite and Amine Agents.

It should readily understood by the disclosure herein that in dosage form of the present invention, the capsule shell comprises at least one primary or secondary amine and optionally a sulfite compound. The fill material of the dosage form optionally comprises either (1) at least one primary or secondary amine, or (2) a sulfite compound; or (3) at least one primary or secondary amine and a sulfite compound. Moreover, it should be understood that “at least one primary or secondary amine” contemplates the presence of one or more primary amines, one or more secondary amines, and combinations of primary and secondary amines.

Other Excipient Fill Material.

Fill material of the present invention optionally further comprises at least one pharmaceutically acceptable free radical-scavenging antioxidant. A free radical-scavenging antioxidant is to be contrasted with a “non-free radical-scavenging antioxidant”, i.e., an antioxidant that does not possess free radical-scavenging properties. Non-limiting illustrative examples of suitable free radical-scavenging antioxidants include α-tocopherol (vitamin E), ascorbic acid (vitamin C) and salts thereof including sodium ascorbate and ascorbic acid palmitate, butylated hydroxyanisole (BHA), butylated hydroxytoluene (BHT), fumaric acid and salts thereof, hypophosphorous acid, malic acid, alkyl gallates, for example propyl gallate, octyl gallate and lauryl gallate, sodium sulfite, sodium bisulfite and sodium metabisulfite. Preferred free radical-scavenging antioxidants are alkyl gallates, vitamin E, BHA and BHT. More preferably the at least one free radical-scavenging antioxidant is propyl gallate.

One or more free radical-scavenging antioxidants are optionally present in dosage forms of the invention in a total amount effective to substantially reduce formation of an addition compound, typically in a total amount of about 0.01% to about 5%, preferably about 0.01% to about 2.5%, and more preferably about 0.01% to about 1%, by weight of the fill material.

Fill material according to the invention optionally comprises one or more pharmaceutically acceptable sweeteners. Non-limiting examples of suitable sweeteners include mannitol, propylene glycol, sodium saccharin, acesulfame K, neotame and aspartame. Alternatively or in addition, a viscous sweetener such as sorbitol solution, syrup (sucrose solution) or high-fructose corn syrup can be used and, in addition to sweetening effects, can also be useful to increase viscosity and to retard sedimentation.

Fill material of the invention optionally comprises one or more pharmaceutically acceptable preservatives other than free radical-scavenging antioxidants. Non-limiting examples of suitable preservatives include benzalkonium chloride, benzethonium chloride, benzyl alcohol, chlorobutanol, phenol, phenylethyl alcohol, phenylmercuric nitrate, thimerosal, etc.

Fill material of the invention optionally comprises one or more pharmaceutically acceptable wetting agents. Surfactants, hydrophilic polymers and certain clays can be useful as wetting agents to aid in dissolution and/or dispersion of a hydrophobic drug such as celecoxib. Non-limiting examples of suitable surfactants include benzalkonium chloride, benzethonium chloride, cetylpyridinium chloride, dioctyl sodium sulfosuccinate, nonoxynol 9, nonoxynol 10, octoxynol 9, poloxamers, polyoxyethylene (8) caprylic/capric mono- and diglycerides (e.g., Labrasol™ of Gattefossé), polyoxyethylene (35) castor oil, polyoxyethylene (20) cetostearyl ether, polyoxyethylene (40) hydrogenated castor oil, polyoxyethylene (10) oleyl ether, polyoxyethylene (40) stearate, polysorbate 20, polysorbate 40, polysorbate 60, polysorbate 80 (e.g., Tween™ 80 of ICI), propylene glycol laurate (e.g., Lauroglycol™ of Gattefossé), sodium lauryl sulfate, sorbitan monolaurate, sorbitan monooleate, sorbitan monopalmitate, sorbitan monostearate, tyloxapol, and mixtures thereof.

Additionally, dosage forms of the invention optionally comprise one or more pharmaceutically acceptable buffering agents, flavoring agents, colorants, stabilizers and/or thickeners. Buffers can be used to control pH of a formulation and can thereby modulate drug solubility. Flavoring agents can enhance patient compliance by making the dosage form more palatable, and colorants can provide a product with a more aesthetic and/or distinctive appearance. Non-limiting examples of suitable colorants include D&C Red No. 33, FD&C Red No. 3, FD&C Red No. 40, D&C Yellow No. 10, and C Yellow No. 6.

Drug Dissolution and Gelatin Cross-Linking.

Without being bound by theory, the inventors believe that gelatin cross-linking can result from a process by which amino acid residues of gelatin covalently bond to form an insoluble material. The process can be the result of low levels of aldehydes coming into contact with the gelatin. Cross-linking of a gelatin capsule can impact product performance by delaying the release of the formulation (containing the active compound) from the capsule shell. The delay in release can, in turn, affect the rate of absorption of the compound into the blood stream and clinical onset of action. While “mild” cross-linking does not necessarily have a significant impact on release of the formulation from the dosage form, “severe” cross-linking can have a significant impact. When cross-linking is severe, it can lead to a delay of release of formulation from the dosage form in humans, potential bioequivalence problems, and a potential delay in clinical onset of action.

Capsules according to the present invention exhibit decreased gelatin cross-linking (and pellicle formation) and, therefore, when filled with a drug-containing composition and placed in an in vitro dissolution assay, are capable of advantageously exhibiting less dissolution rate change during storage under stressed conditions than conventional capsules. Capsules according to the present invention are also believed to exhibit more uniform inter-capsule drug dissolution rate than standard gelatin capsules.

In one embodiment of the present invention, upon (a) filling two or more capsules of the instant invention with a fill material wherein the fill material optionally comprises a drug and at least one substance that promotes cross-linking of gelatin when in contact therewith (the substance being the drug itself or an excipient substance, and the substance acting independently or in combination with one or more other substances to promote said cross-linking); (b) sealing the capsules; (c) immediately testing a first sealed capsule in a first in vitro dissolution assay; (d) storing a second sealed capsule in a closed container maintained at 40° C. and 75% relative humidity for a period of four weeks and, after said storage; (e) testing the second sealed capsule in a second in vitro dissolution assay which is identical to the first in vitro dissolution assay; the amount of drug dissolved at 45 minutes in the second dissolution assay is within ±15 percent and preferably within ±10 percent of the amount of drug dissolved at 45 minutes in the first dissolution assay.

Because gelatin cross-linking may lead to delayed dissolution, storage time-dependent delays in dissolution profile may be a good indicator of gelatin cross-linking during such storage. There are a number of in vitro dissolution assays suitable for determining dissolution profile. Indeed, one skilled in the art is able to design additional assays or modifications thereof. Two dissolution-type test methods were developed and set forth herein and designated the “Tier I” and “Tier II” tests.

In the Tier I test, a gelatin capsule dosage form is placed in a USP apparatus II with a rotating paddle with a paddle speed of 50 rpm in 900 mL of 0.01N HCl+1% Tween 80. Samples are typically withdrawn at 15, 30, 45, 60 and 90 minutes and assayed for drug content by HPLC.

The Tier II test employs the addition of the enzyme pepsin to the media. Pepsin in the human stomach digests cross-linked gelatin. The appropriate amount of pepsin added to the media (750,000 units/L) was determined and reported in Collaborative Development of Two-Tier Dissolution Testing for Gelatin Capsules and Gelatin-Coated Tablets using Enzyme-Containing Media, Stimuli to the Revision Process, Pharmacopeial Forum, Vol. 25, No. 5, September-October 1998. The Tier II drug release test designed in this way is expected to produce a drug release profile that is a reasonable approximation of the drug release profile in humans.

An ‘initial’ drug release profile is determined for each dosage form within a reasonably short time after formation (i.e. dosage form before the formulation is exposed to conditions which might result in gelatin cross-linking, such as temperature or relative humidity). A subsequent profile is determined for samples pulled at subsequent time points. A change from initial to subsequent Tier I profile (i.e. a delay in dissolution) is presumptively attributed to gelatin cross-linking. When such a change is reduced in the Tier II assay (containing pepsin), this reduction is deemed further evidence of gelatin cross-linking upon storage.

### EXAMPLES

The following non-limiting examples are provided for illustrative purposes only and are not to be construed as limitations.

### Example 1

A liquid fill formulation, F0, is prepared as shown in Table 1.

TABLE 1
Liquid fill formulation F0 (mg/g)

| Component | F0 |
|---|---|
| Celecoxib | 278 |
| PEG400 | 335 |
| Tween80 | 197 |
| Oleic Acid | 80 |
| Hydroxypropyl methylcellulose (“HPMC”) | 74 |
| Propyl gallate | 2 |
| Dimethylamino-ethanol (“DMAE”) | 34 |
| Total | 1000 |

### Example 2

Several compositions suitable for preparation of a capsule wall, C1-C14, are prepared as shown in Tables 2 and 3 according to the following procedure. Gelatin and one or primary and/or secondary amines are admixed together to form a dry mixture. One or more plasticizers (glycerol and/or sorbitol) and water are then added to the mixture to form a liquid mixture. The liquid mixture is melted at 80° C. for up to four hours to form a melt. The melt is cooled to 60° C. to form a flowable gelatin mix and is fed into the two spreader boxes of a rotary die soft gelatin capsule manufacturing machine, which controls the flow of said mix onto two air-cooled rotating drums, where two white opaque gelatin ribbons are cast and further processed to white opaque, soft gelatin capsules, at a rate of about 15,000 capsules per hour; capsules are filled with 1 ml of fill Formulation F0 of Example 1. The capsules are then dried in a tumbler dryer with an air blast at 21 C and 20% relative humidity, and are then brought to room temperature.

TABLE 2
Compositions C1-C7 for preparing a capsule wall (% wt)

| Component | C1 | C2 | C3 | C4 | C5 | C6 | C7 |
|---|---|---|---|---|---|---|---|
| Gelatin | 40 | 42 | 45 | 50 | 35 | 40 | 41 |
| Glycerol, 85% | 25 | 10 | 7.5 | — | 10 | 10 | 11 |
| Sorbitol | — | 15 | 10 | 25 | 10 | 10 | 9 |
| Tromethamine | 10 | 7.5 | 4 | 5 | 3 | 6 | 0.5 |
| Water | 25 | 25.5 | 33.5 | 20 | 42 | 34 | 38.5 |

TABLE 3
Compositions C8-C14 for preparing a capsule wall (% wt)

| Component | C8 | C9 | C10 | C11 | C12 | C13 | C14 |
|---|---|---|---|---|---|---|---|
| Gelatin | 40 | 42 | 45 | 50 | 35 | 40 | 41 |
| Glycerol, 85% | 25 | 10 | 7.5 | — | 10 | 10 | 11 |
| Sorbitol | — | 15 | 10 | 25 | 10 | 10 | 9 |
| Diethanolamine | 5 | 7.5 | 4 | 2.5 | 0.5 | 6 | 5 |
| Tromethamine | 5 | — | 2 | 2.5 | — | — | — |
| Water | 25 | 25.5 | 31.5 | 20 | 44.5 | 34 | 34 |

Several comparator compositions (no amine compound), CC1-CC7, are prepared as described immediately above having compositions shown in Table 4.

TABLE 4
Comparator compositions CC1-CC7 for preparing a
capsule wall (% wt)

| Component | CC1 | CC2 | CC3 | CC4 | CC5 | CC6 | CC7 |
|---|---|---|---|---|---|---|---|
| Gelatin | 40 | 42 | 45 | 50 | 35 | 40 | 41 |
| Glycerol, 85% | 25 | 10 | 7.5 | — | 10 | 10 | 11 |
| Sorbitol | — | 15 | 10 | 25 | 10 | 10 | 9 |
| Water | 35 | 33 | 37.5 | 25 | 45 | 40 | 39 |

Filled capsules are stored 40 ° C. and 75% relative humidity for up to 24 weeks. After 24 weeks of storage, each of the capsules are analyzed for pellicle formation. Overall, capsules prepared from compositions C1-C14 (comprising a primary amine) exhibit less pellicle formation than do capsules prepared from comparative compositions CC-1-CC7 (no primary amine).

### Example 3

Three fill formulations, F1-F3, were prepared as shown in Table 5. One ml of each fill formulation was filled into each of several standard (no primary or secondary amine) soft gelatin capsules (R. P. Scherer).

TABLE 5
Composition of fill formulations F1-F3

| Component | F1 | F2 | F3 |
|---|---|---|---|
| Celecoxib | 200 | 278 | 270 |
| PEG400 | 271 | 337 | 334 |
| Tween80 | 217 | 195 | 194 |
| Oleic Acid | 61 | 80 | 78 |
| PVP | 47 | — | — |
| Ethanol | 113 | — | — |
| HPMC | 38 | 74 | 74 |
| Water | 26 | — | 10 |
| Propyl gallate | 1 | 2 | 2 |
| Tromethamine | 26 | — | 5 |
| DMAE | — | 34 | 33 |
| Total | 1000 | 1000 | 1000 |

Filled caspsules were placed in a sealed container and store at 40° C. and 75% relative humidity for a period of up to 24 weeks. At various times during storage, capsules were removed from the closed container and evaluated, by visual inspection, for presence or absence of pellicle formation (i.e., cross-linking). Each evaluated capsule was assigned a numerical indicator based on any pellicle observed according to the following scale: (1)=no pellicle; (2)=thin, incomplete pellicle; (3)=thin, complete pellicle; (4)=strong, complete pellicle which inhibits compression of capsule; and (5) thick, strong, and severe pellicle. Pellicle formation observations are shown in Table 6.

TABLE 6
Pellicle formation after storage for up to 24 weeks
at 40° C. and 75% relative humidity

| Time | | | |
|---|---|---|---|
| (weeks) | F1 | F2 | F3 |
| 0 | 1 | 1 | 1 |
| 2 | — | 3 | 1 |
| 4 | 1 | 3 | 2 |
| 6 | — | 3 | 3 |
| 8 | 1 | 4 | 3 |
| 12 | 1 | — | — |
| 24 | 1 | — | — |

As shown in Table 6, capsules containing fill formulation F1 (comprising tromethamine in an amount of about 3% by weight of the fill material) exhibited no pellicle formation during storage for a period of six months. By contrast, capsules containing Fill Formulation F2 (no primary or secondary amine compound) or F3 (0.5% tromethamine) exhibited pellicle formation by 15 and 30 days of storage, respectively.

### Example 4

A composition of the invention is prepared by mixing in a vessel (a) 35% B grade, 150 Bloom strength, pharmaceutical grade gelatin; (b) 15% chilled glycerol; (c) 5% tromethamine; and (d) 45% chilled deionized water. The mixture is melted at 80° C. for up to four hours to form a melt. The melt is cooled to 60° C. to form a flowable gelatin mix and is fed into the two spreader boxes of a rotary die soft gelatin capsule manufacturing machine, which controls the flow of said mix onto two air-cooled rotating drums, where two white opaque gelatin ribbons are cast and further processed to white opaque, soft gelatin capsules, at a rate of about 15,000 capsules per hour. The capsules are then dried in a tumbler dryer with an air blast at 21 C.° and 20% relative humidity, and are then brought to room temperature.

### Example 5

Several compositions suitable for preparation of a capsule wall, C15-C21, are prepared as shown in Tables 7, according to the procedure described in Examples 1 and 2.

TABLE 7
Compositions C15-C21 for preparing a capsule wall (% wt)

| Component | C15 | C16 | C17 | C18 | C19 | C20 | C21 |
|---|---|---|---|---|---|---|---|
| Gelatin | 40 | 42 | 45 | 50 | 35 | 40 | 41 |
| Glycerol, 85% | 25 | 10 | 7.5 | — | 10 | 10 | 11 |
| Sorbitol | — | 15 | 10 | 25 | 10 | 10 | 9 |
| Sodium | 5 | 7.5 | 4 | 2.5 | 0.5 | 6 | 5 |
| metabisulfite | | | | | | | |
| Tromethamine | 5 | — | 2 | 2.5 | — | — | — |
| Water | 25 | 25.5 | 31.5 | 20 | 44.5 | 34 | 34 |

### Example 6

The cross-linking behavior of two soft gelatin formulations was investigated over a 6 month period. As shown below (Table 7), Formulation 30 (the control lot) contains dimethylaminoethanol (“DMAE”) and no sulfite. Formulation 19 (the test lot) was similar to the Formulation 30, except that Formulation 19 additionally comprises sodium metabisulfite in the fill material.

TABLE 7
Fill material of Formulations 30 and 19 (mg/g)

| Component | Formulation 30 | Formulation 19 |
|---|---|---|
| celecoxib | 278 | 270 |
| PEG 400 | 337 | 335 |
| Tween 80 | 195 | 195 |
| oleic acid | 80 | 78 |
| HPMC | 74 | 74 |
| DMAE | 34 | 35 |
| propyl gallate | 2 | 2 |
| water | — | 7 |
| sodium metabisulfite | — | 4 |

Both soft gelatin capsule formulations were placed into non-induction-sealed hydroxypropyl ethylene bottles and stored at either 25° C. and 60% RH or 40° C. and 75% RH. Using such bottles, RH inside the bottles readily equilibrates with the RH outside of the bottles (60% or 75%). Periodically, capsules were tested for degree of cross-linking of the soft gelatin samples as estimated by the drug release profile.

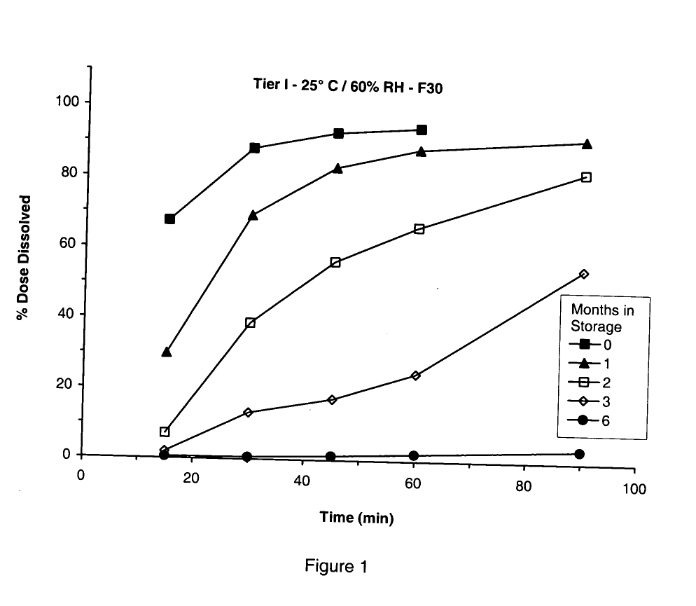

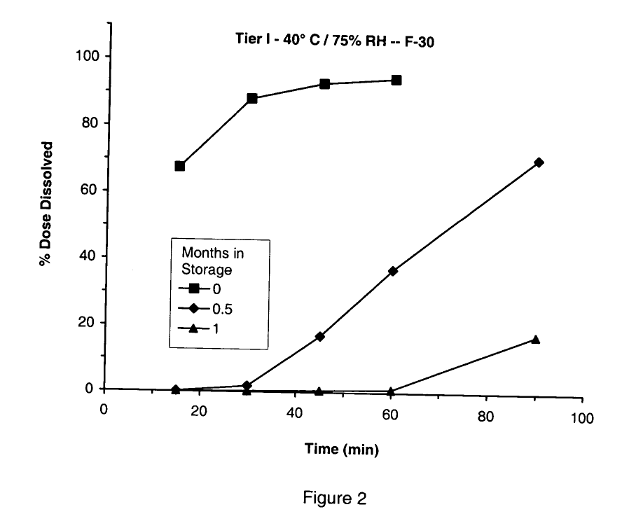

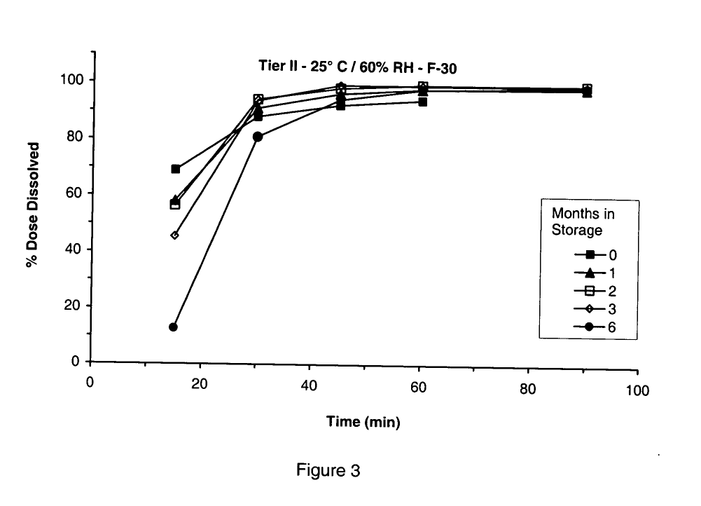

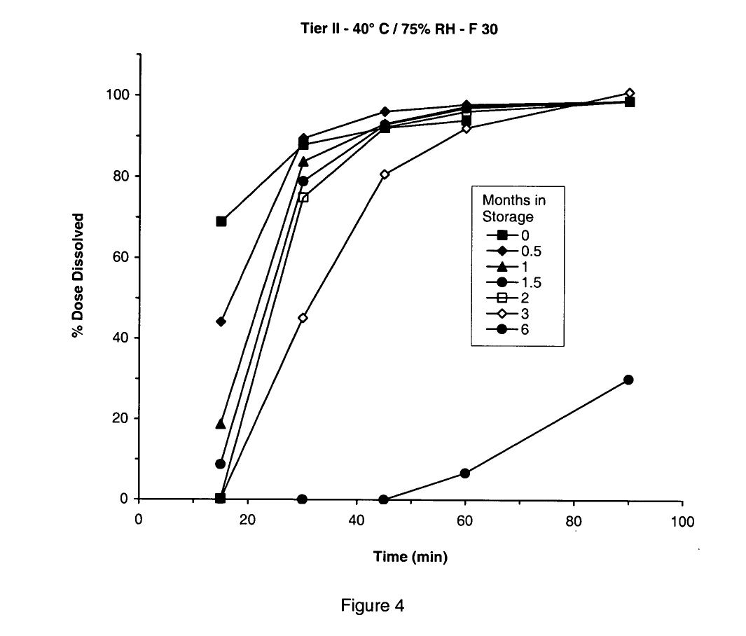

Formulation 30. The Tier I drug release results for control Formulation 30 at 25° C./60% relative humidity (“RH”) and 40° C./75% RH are shown in FIGS. 1 and 2 and the Tier II drug release results for the same lot and conditions are shown in FIGS. 3 and 4. As early as 1 month of storage, there was a marked delay in the Tier I drug release profile at both temperature conditions. This delay increased with storage time. The Tier II drug release profile at 25° C./60% RH and at 40° C./75% RH shows a significant but markedly reduced delay in release profile.

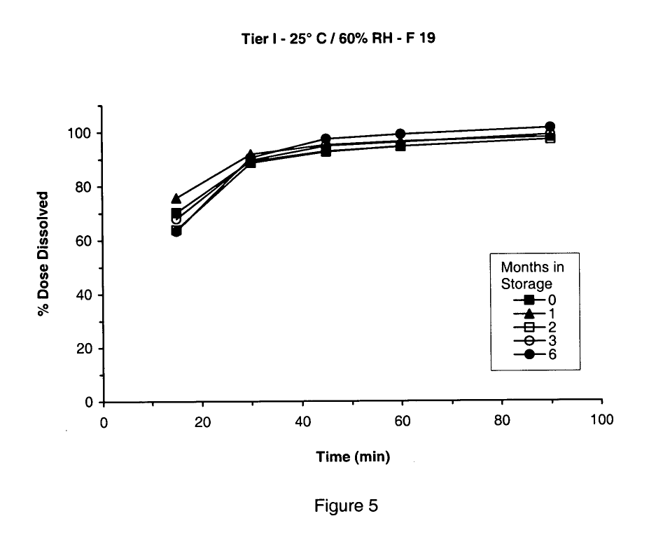

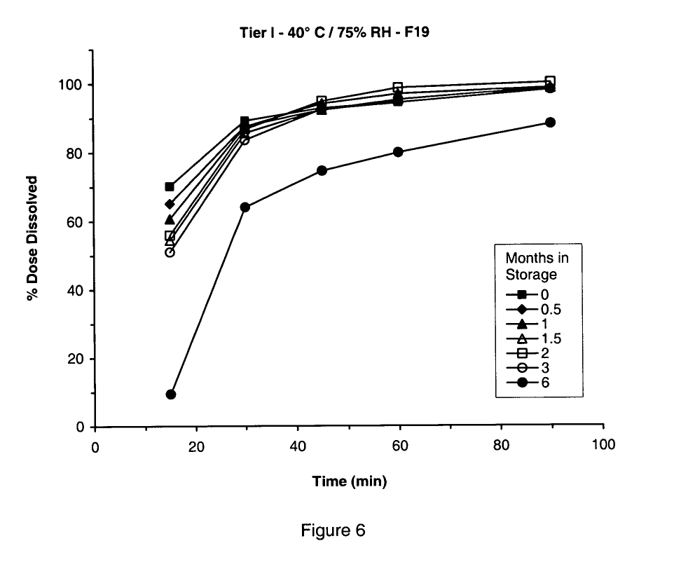

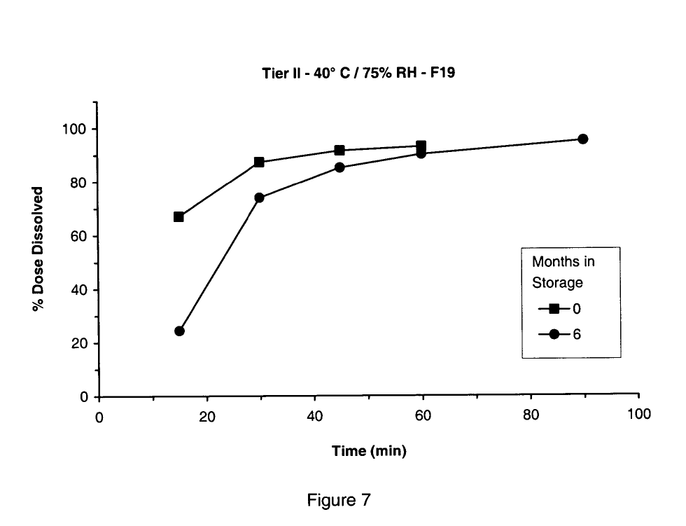

Formulation 19. The Formulation 19 Tier I drug release results for the 25° C/60% RH condition are shown in FIG. 5. No change in the drug release profile is observed through 6 months, indicating that no cross-linking has occurred. Accordingly, the analogous Tier II test for this sample was not performed. FIG. 6 displays the Tier I results for Formulation 19 at 40° C./75% RH. No change in drug release profile is observed for most of the stability time points with the exception of the 6 month time point. To determine if the change in drug release profile at 6 months is a result of cross-linking, the Tier II test was performed on this sample. The Tier II results are displayed in FIG. 7. The Tier I and Tier II results are very similar for this 6 month sample indicating that the change in drug release profile is not attributable to cross-linking.

These data indicate that there was severe cross-linking observed in the Formulation 30. The change in the Tier II drug release profile (i.e. reduced delay) indicates that Tier I delayed release is the result of cross-linking for this formulation and further indicates that a significant delay in the drug release profile in humans would be likely. The Formulation 19, containing sodium metabisulfite, exhibits no measurable cross-linking through 6 months at stringent (40° C./75% RH) storage conditions. These data demonstrate that the addition of sodium metabisulfite to this formulation significantly reduces the rate of cross-linking and indeed may inhibit cross-linking completely. Without being bound by theory, sodium metabisulfite is believed to inhibit cross-linking by a process in which sodium metabisulfite reacts with aldehydes forming a bisulfite addition product. Thus, sodium metabisulfite may effectively scavenges aldehydes making them unavailable to promote cross-linking in the gelatin.

### Example 7

Four soft gelatin Celecoxib formulations were prepared as shown in Table 8 and tested for pellicle formation at 40° C. and 75% relative humidity (“RH”).

In absence of sulfite, complete pellicle formation was apparent after only 2 weeks storage at 40° C./75% RH (Formulation 30; cross-linking rating=3).

At a Tris concentration of 5 mg/g in the formulation (Formulation 20), delayed pellicle formation but was insufficient to prevent a complete pellicle formation (the cross-linking rating=3) upon 1.5 months storage under 40° C./75% RH.

At a higher Tris concentration in the formulation (26 mg/g, Formulation 50), gelatin cross-linking is completely prevented upon 6 months storage under 40° C./75% RH.

A low sodium metabisulfite (SMB) concentration of 4 mg/g in the formulation (Formulation 19) appeared sufficient to prevent the pellicle formation upon 2 months storage under 40° C./75% RH.

TABLE 8
Gelatin cross-linking analysis of soft gelatin at
40° C./75% RH storage

| Months | | | | | | | | |
|---|---|---|---|---|---|---|---|---|
| at 40° C./ | | | | | | | | |
| 75% | Formulation | | Formulation | | Formulation | | Formulation | |
| RH | 50 mg/ml | | 30 mg/ml | | 19 mg/ml | | 20 mg/ml | |
| | Celecoxib | 200 | Celecoxib | 278 | Celecoxib | 270 | Celecoxib | 270 |
| | PEG400 | 271 | PEG400 | 337 | PEG400 | 335 | PEG400 | 334 |
| | Tween80 | 217 | Tween80 | 195 | Tween80 | 195 | Tween80 | 194 |
| | Oleic acid | 61 | Oleic acid | 80 | Oleic acid | 78 | Oleic acid | 78 |
| | PVP | 47 | | | | | | |
| | EtOH | 113 | | | | | | |
| | HPMC | 38 | HPMC | 74 | HPMC | 74 | HPMC | 74 |
| | | | DMAE | 34 | DMAE | 35 | DMAE | 33 |
| | propyl gallate | 1 | propyl gallate | 2 | propyl gallate | 2 | propyl gallate | 2 |
| | water | 26 | | | water | 7 | water | 10 |
| | Tris | 26 | | | SMB | 4 | Tris | 5 |

| | | | | |
|---|---|---|---|---|
| 0 | 1 | 1 | 1 | 1 |
| 0.5 | | 3 | 1 | 1 |
| 1 | 1 | 3 | 1 | 2 |
| 1.5 | | 3 | 1 | 3 |
| 2 | 1 | 4 | 1 | 3 |
| 3 | 1 | | | |
| 6 | 1 | | | |

### Example 8

In order to gain insight in to the mechanism by which Tris (hydroxymethyl aminomethane) in fill material of a gelatin capsule prevents pellicle formation, a dosage form (of Formulation X-60 set forth in Table 9) was prepared and stored under two different conditions as shown in Table 10. At the times indicated, capsules were removed and Tris content was quantified in the fill material and in the capsule. As shown in Table 10, upon storage with time, Tris content in the capsules increased and Tris content in the fill material decreased in comparison to the initial formulation.

TABLE 9
Soft gelatin capsule Formulation X-60

| Ingredient | Formulation X-60 |
|---|---|
| Celecoxib | 200 |
| PEG 400 | 271 |
| Tween 80 | 217 |
| Oleic acid | 61 |
| Tris | 26 |
| Water | 26 |
| Propyl gallate | 1 |
| PVP-12PF | 47 |
| Abs. EtOH | 113 |
| HPMC-E5 | 38 |
| Total | 1000 mg/g |
| Fill Volume | 0.92 mL |
| (200 mg drug) | |
| Dosage Form | 18 Oblong |
| | soft gelatin capsule |

TABLE 10
Tris content in capsule shells following
storage of Formulation X-60

| Soft gelatin capsule | Tris in fill | Tris in shell |
|---|---|---|
| Storage conditions | (mg) | (mg) |
| 25° C./60% RH | | |
| T = 2 months | 18.7 | 5.3 |
| T = 6 months | 17.9 | 6.0 |
| T = 8 months | 16.4 | 6.5 |
| T = 10 months | 17.6 | 7.0 |
| 40° C./75% RH | | |
| T = 2 months | 13.5 | 10.5 |
| T = 6 months | 10.8 | 11.1 |
| T = 8 months | 10.0 | 10.6 |
| T = 10 months | 10.0 | 13.3 |

26 mg Tris in a soft gelatin capsule at T = 0

What is claimed is:

1. A composition comprising gelatin and an amine agent that comprises at least one pharmaceutically acceptable primary or secondary amine, the composition being suitable for preparation of a pharmaceutical capsule shell.

2. The composition of claim 1 wherein the amine agent is present in an amount effective to inhibit cross-linking of the gelatin and/or pellicle formation in a capsule shell prepared from the composition.

3. The composition of claim 1 wherein the amine agent comprises a compound selected from the group consisting of tromethamines, ethanolamine, ethylenediamine, diethylamine, ethylene N-methyl-D-glucamine, amino acids, diethanolamine, benethamine, benzathine, piperazine, hydrabamine, and imidazoles.

4. The composition of claim 1 wherein the amine agent is present in an amount of not more than about 10% of the composition on a dry weight basis.

5. The composition of claim 1 wherein the amine agent is present in an amount of not more than about 5% of the composition on a dry weight basis.

6. The composition of claim 1 wherein the amine agent is present in an amount of not more than about 2% of the composition on a dry weight basis.

7. The composition of claim 1 further comprising at least one excipient selected from the group consisting of decomposition inhibitors, opacifying agents, preservatives and plasticizers.

8. The composition of claim 1 further comprising a plasticizer selected from the group consisting of polyhydroxy-alcohols, esters of polyhydroxy-alcohols, dialkylphthalates, lower alkyl citrates wherein the lower alkyl has 1-6 carbon atoms, glycols, polyglycols, ricinoleic acid and ricinoleic acid esters.

9. The composition of claim 1 further comprising a plasticizer selected from the group consisting of sorbitol, glycerol, propylene glycols and polyethylene glycols.

10. The composition of claim 1 further comprising a preservative selected from the group consisting of methylparabens, propylparabens, butylparabens, sorbic acid, benzoic acid, editic acids, phenolic acids, sorbates, and propionates.

11. The composition of claim 1 further comprising titanium dioxide.

12. The composition of claim 1 further comprising sulfur dioxide.

13. The composition of claim 1 that is in a form of capsule shells.

14. The composition of claim 13 wherein each of said capsule shells defines a fill volume.

15. The composition of claim 13 wherein the capsule shells are soft gelatin capsule shells.

16. The composition of claim 14 wherein the fill volume has a capacity of about 0.1 ml to about 2 ml.

17. The composition of claim 16 wherein the fill volume has a capacity of not more than about 1 ml.

18. The composition of claim 14 wherein the capsule shells are suitable for oral delivery of a drug contained in the fill volume.

19. A pharmaceutical dosage form comprising a fill material sealed in capsule shells, wherein the capsule shells comprise gelatin and an amine agent that comprises at least one pharmaceutically acceptable primary or secondary amine, and wherein said amine agent is present in an amount sufficient to inhibit gelatin cross-linking and/or pellicle formation in the gelatin capsule shells upon storage of the dosage form.

20. The dosage form of claim 19 wherein the fill material is liquid.

21. The dosage form of claim 20 wherein the fill material is self-emulsifying upon contact with gastric fluid.

22. The dosage form of claim 19 wherein the fill material comprises an amine agent comprising at least one pharmaceutically acceptable primary or secondary amine wherein the amine agent in the fill material is present in an amount effective, in combination with the amine agent in the capsule shell, to inhibit gelatin cross-linking and/or pellicle formation in the capsule shell upon storage of the dosage form.

23. The dosage form of claim 19 wherein the fill material comprises a pharmaceutically acceptable sulfite compound in an amount effective, in combination with the amine agent in the capsule shell, to inhibit gelatin cross-linking and/or pellicle formation in the capsule shell upon storage of the dosage form.

24. The dosage form of claim 19 wherein the fill material comprises a drug.

25. The dosage form of claim 24 wherein the drug is of low water solubility.

26. The dosage form of claim 24 wherein the drug is a selective cyclooxygenase-2 inhibitory drug.

27. The dosage form of claim 26 wherein the selective cyclooxygenase-2 inhibitory drug is a compound of formula (I) 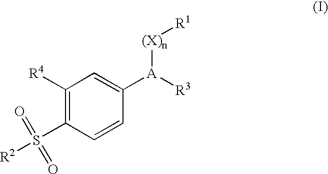
wherein:
A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;
X is O, S or CH₂;
n is 0 or 1;
R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is methyl, amino or aminocarbonylalkyl;
R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R⁴ is selected from hydrido and halo.

28. The dosage form of claim 26 wherein the selective cyclooxygenase-2 inhibitory drug is selected from the group consisting of celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, and pharmaceutically acceptable salts and prodrugs thereof.

29. The dosage form of claim 26 wherein the selective cyclooxygenase-2 inhibitory drug is celecoxib.

30. The dosage form of claim 24 wherein the fill material further comprises at least one substance that promotes cross-linking of gelatin when in contact therewith, said substance being the drug or an excipient substance, and said substance acting independently or in combination with one or more other substances to promote said cross-linking.

31. The dosage form of claim 30 comprising a first and a second of said capsule shells, said first and second capsule shells being substantially identical; wherein upon
(a) testing a first capsule shell in a first in vitro dissolution assay;
(b) storing a second capsule shell in a closed container maintained at 40° C. and 85% relative humidity for a period of four weeks; and, after said storage,
(c) testing the second sealed capsule shell in a second in vitro dissolution assay which is substantially identical to the first in vitro dissolution assay; the amount of drug dissolved at 45 minutes in the second dissolution assay is within ±15 percent of the amount of drug dissolved at 45 minutes in the first dissolution assay; and wherein the first in vitro dissolution assay is conducted within a reasonably short time after preparation of the composition.
